(19) United States
(12) Patent Application Publication
(10) Pub. No.: US 2022/0072008 A1
(43) Pub. Date: Mar. 10, 2022

(54) METHOD OF USING PROGESTERONE RECEPTOR AGONISTS FOR THE TREATMENT OF COVID-19

(71) Applicant: CEDARS-SINAI MEDICAL CENTER, Los Angeles, CA (US)

(72) Inventors: Sara Ghandehari, Los Angeles, CA (US); Samuel Pepkowitz, Los Angeles, CA (US)

(73) Assignee: CEDARS-SINAI MEDICAL CENTER, Los Angeles, CA (US)

(21) Appl. No.: 17/469,089

(22) Filed: Sep. 8, 2021

Related U.S. Application Data
(60) Provisional application No. 63/075,511, filed on Sep. 8, 2020.

(51) Int. Cl. A61K 31/57; A61K 45/06; A61P 31/14

(52) U.S. Cl. CPC A61K 31/57; A61P 31/14; A61K 45/06

(57) ABSTRACT

The invention describes methods of treating COVID-19, particularly treating men who have COVID-19. The treatment includes administering a progesterone receptor agonist, such as progesterone to a subject who has one or more symptoms of COVID-19, or who is confirmed to have COVID-19.

### CROSS-REFERENCE TO RELATED APPLICATIONS

This application includes a claim of priority under 35 U.S.C. § 119(e) to U.S. provisional patent application No. 63/075,511, filed Sep. 8, 2020, the entirety of which is hereby incorporated by reference.

### FIELD OF INVENTION

This invention relates to the treatment of COVID-19, and particularly, in men.

### STATEMENT REGARDING PRIOR DISCLOSURE BY INVENTOR OR JOINT INVENTOR

The following are grace period inventor disclosures by one or more joint inventors: (1) ClinicalTrials.gov Identifier: NCT04365127, first version posted Apr. 28, 2000; (2) Progesterone in Addition to Standard of Care vs Standard of Care Alone in the Treatment of Men Hospitalized With Moderate to Severe COVID-19 A Randomized, Controlled Pilot, Trial CHEST INFECTIONS: ORIGINAL RESEARCH| VOLUME 160, IS SUE 1, P74-84, Jul. 1, 2021, published online Feb. 20, 2021.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

As of early September 2020, there are over 27 million cases of COVID-19 causing over 800,000 deaths world-wide. As of January 2021, more than 96 million cases of COVID-19 with 2 million deaths have been reported; of these, men with severe illness appear to be disproportionately overrepresented, with some data suggesting that for every 10 women who are hospitalized, admitted to the ICU, or die of COVID-19, 12 men are hospitalized, 19 men are admitted to ICU, and 15 men die. This sex disparity is attributable in part to the higher prevalence of preexisting comorbidities associated with worse COVID-19 outcomes among men. Men are more likely to engage in smoking and alcohol use, with greater reluctance to seek health care, which may promote poorly controlled chronic conditions. At a biological level, differences in gene expression and hormonal influences may favor the female sex as it relates to the course of this disease.

As such, there is an urgent need in the art for therapies against COVID-19, and particularly for therapies for men who have a higher risk for mortality and/or severe symptoms.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of treating coronavirus disease 2019 (COVID-19) in a subject in need thereof, comprising: administering a quantity of a progesterone receptor agonist to the subject.

In various embodiments, the progesterone receptor agonist can be selected from the group consisting of progesterone, levonorgestrel, etonogestrel, hydroxyprogesterone caproate, ulipristal acetate, medroxyprogesterone acetate, norethindrone, desogestrel, chlormadinone acetate, progesterone, dienogest, drospirenone, dydrogesterone, everolimus, hydroxyprogesterone, lynestrenol, medrogestone, medroxyprogesterone, megestrol acetate, nomegestrol acetate, norgestrel, promegestone, vilaprisan, danazol, methylestrenolone, VOLT-02, EC-313, MDC-WWM, and combinations thereof.

In various embodiments, the quantity of the progesterone receptor agonist can be about 50 mg to about 150 mg.

In various embodiments, the progesterone receptor agonist can be progesterone. In various embodiments, the quantity of progesterone can be about 100 mg.

In various embodiments, the progesterone receptor agonist can be administered one to four times daily. In various embodiments, the progesterone receptor agonist can be administered twice daily.

In various embodiments, the progesterone receptor agonist can be administered subcutaneously. In various embodiments, the progesterone receptor agonist can be administered subcutaneously, twice daily for five days.

In various embodiments, the progesterone receptor agonist can be administered in addition to one or more standard-of-care therapy for COVID-19.

In various embodiments, the subject can be male.

In various embodiments, the subject can have or can be suspected to have a baseline serum progesterone level of less than 1 ng/mL. In various embodiments, the subject can have or can be suspected to have a progesterone level of less than 20 ng/mL.

In various embodiments, after administration of the progesterone receptor agonist, the subject can have a progesterone level of 11.1 ng/mL to 290 ng/mL.

In various embodiments, the subject can be male who has one or more symptoms of COVID-19, can be confirmed to have COVID-19, can be hospitalized or combinations thereof.

In various embodiments, administering the progesterone receptor agonist comprises subcutaneously administering about 100 mg of progesterone twice a day to a male subject who has one or more symptoms of COVID-19, or who is confirmed to have COVID-19.

In various embodiments, the method can further comprise administering a concomitant therapeutic intervention selected from the group consisting of azithromycin, remdesivir, systemic glucocorticoid, tocilizumab, convalescent plasma, hydroxychloroquine, monoclonal antibodies against SARS-CoV-2, baricitinib, anticoagulant, and combinations thereof. In various embodiments, the systemic glucocorticoid can be dexamethasone, prednisone, or methylprednisolone. In various embodiments, the monoclonal antibodies against SARS-CoV-2 can be casirivimab, imdevimab, bamlanivimab, etesevimab, sotrovimab, or combinations thereof, or wherein the monoclonal antibodies against SARS-CoV-2 is a combination of casirivimab and imdevimab, or a combination of bamlanivimab and etesevimab.

Various embodiments of the present invention provide for a method of treating coronavirus disease 2019 (COVID-19) in a male subject in need thereof, comprising: administering about 100 mg of progesterone, twice daily to the male subject who has one or more symptoms of COVID-19, or who is confirmed to have COVID-19. In various embodiments, administering is subcutaneously administering. In various embodiments, the progesterone is administered for about five days.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced FIGURES. It is intended that the embodiments and FIGURES disclosed herein are to be considered illustrative rather than restrictive.

FIG. 1 depicts cumulative probability of improvement or discharge on day 7. During the first seven study days, the cumulative probability of clinical improvement (an increase of at least one point on the seven-point scale or live discharge) was significantly higher in the progesterone group, 0.76 (95% CI, 0.55-0.93) vs 0.55 (95% CI, 0.28-0.68) in the control group (log-rank P=0.014), by Kaplan-Meier estimation. One patient in the progesterone group showed improvement on day 2 but was subsequently noncompliant with study protocols and was transferred to another facility. For the purpose of this analysis, this patient was excluded.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Therapeutically effective amount” as used herein refers to that amount which is capable of achieving beneficial results in a patient with COVID-19. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease even if the treatment is ultimately unsuccessful.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In some embodiments, the subject is a human. In some embodiments, the subject is a male subject. In some embodiments, the subject has COVID-19.

A subject may be one who has been previously diagnosed with or identified as suffering from or having a disease, disorder or condition in need of treatment or one or more complications related to the disease, disorder, or condition, and optionally, have already undergone treatment for the disease, disorder, or condition or the one or more complications related to the disease, disorder, or condition. Alternatively, a subject can also be one who has not been previously diagnosed as having a disease, disorder, or condition or one or more complications related to the disease, disorder, or condition. For example, a subject may be one who exhibits one or more risk factors for a disease, disorder, or condition or one or more complications related to the disease, disorder, or condition or a subject who does not exhibit risk factors. A “subject in need” of treatment for a particular disease, disorder, or condition may be a subject suspected of having that disease, disorder, or condition, diagnosed as having that disease, disorder, or condition, already treated or being treated for that disease, disorder, or condition, not treated for that disease, disorder, or condition, or at risk of developing that disease, disorder, or condition.

A recent review article discusses the diverse cellularly-based immune responses that impact the innate and classical immune responses to infection. For example, progesterone (P4) can inhibit innate immune response by several pathways that includes gene transcription of the NF-KB pathway as well as the cyclooxygenase 2 pathway to decrease TNF, INFγ, IL-1β, and other inflammatory cytokines as well as to augment IL-10 (an anti-inflammatory cytokine). Further impact on the immune response to infection include modulation of macrophage phenotype to an anti-inflammatory M2 state, downregulation of macrophage nitric oxide production, skew TH1 responses to TH2, reduce NK cells, and increase T regs.

Progesterone is widely used for a variety of indications in the form of oral and intramuscular preparations. In a few clinical trials, intravenous (IV) progesterone has been used in severe traumatic brain injury. While the results were mixed on therapeutic benefit in this setting, IV progesterone was found to have a favorable safety profile.

Described herein, we evaluated the clinical safety and efficacy of progesterone administration to men with confirmed COVID-19 through a randomized controlled trial as described in the examples. We believed that the anti-inflammatory properties of progesterone can dampen the systemic cytokine response and offer therapeutic benefit in patients suspected or confirmed to have COVID-19. This in turn led to reducing severity of illness, length of hospital stays, need for intubation and mechanical ventilation, as well as death. The results described herein show greater degree of improvement in clinical status at Day 7 for subjects who received progesterone compared to those on standard of care (SOC) (see e.g., example 2).

Forty-two patients were enrolled from April 2020 to August 2020; 22 were randomized to the control group and 20 to the progesterone group. Two patients from the progesterone group withdrew from the study before receiving progesterone. There was a 1.5-point overall improvement in median clinical status score on a seven-point ordinal scale from baseline to day 7 in patients in the progesterone group as compared with control subjects (95% CI, 0.0-2.0; P=0.024). There were no serious adverse events attributable to progesterone.

Patients treated with progesterone required three fewer days of supplemental oxygen (median, 4.5 vs 7.5 days) and were hospitalized for 2.5 fewer days (median, 7.0 vs 9.5 days) as compared with control subjects. While not wishing to be bound by any particular theory, the inventors believe that progesterone; for example, at a dose of 100 mg, twice daily by subcutaneous injection in addition to SOC, represent a safe and effective approach for treatment in hypoxemic men with moderate to severe COVID-19.

The study results described herein shows that the use of progesterone, in addition to SOC treatment measures in hospitalized men with COVID-19 who are hypoxemic, could lead to improved clinical outcomes with minimal safety concerns. We noted that addition of progesterone to SOC treatment was associated with improved clinical status assessed on a seven-point ordinal scale, a trend toward fewer days on supplemental oxygen, reduced need for mechanical ventilation, and reduced length of hospital stay.

The sex difference in illness severity and mortality outcomes in COVID-19, as well as in prior coronavirus outbreaks, has been demonstrated in multiple populations. The concept of a less effective immune response to viral infections as a consequence of differences in sex hormones between men and women has been described previously and may be related to unequal endogenous progesterone levels, a steroid hormone with well-described antiinflammatory properties. The corpus luteum produces progesterone in women with peak levels (10-20 ng/mL) during the luteal phase of the menstrual cycle. Adrenal glands and testes produce progesterone in men, but at much lower concentrations (0.13-0.97 ng/mL), similar to those of postmenopausal women. The role of progesterone extends beyond fertility and menstruation.

It binds to glucocorticoid receptors, and indeed most immune cells express progesterone receptors. It is possible that higher endogenous levels of progesterone protect women from progressing to severe illness in COVID-19.

A major driver of morbidity and mortality in COVID-19 is the exuberant inflammatory response, sometimes termed a “cytokine storm,” mediated by production of proinflammatory cytokines (IL-6, IL-1b, tumor necrosis factor-a) and macrophage hyperactivation. Previous preclinical and clinical studies have demonstrated that the elevated concentrations of estrogen and progesterone in women are associated with inflammatory response attenuation through IL-1b and IL-12 inhibition, decreased T-cell IL-6 receptor expression, and a bias toward helper T-cell type 2 production, which secrete IL-4, IL-5, IL-10, and other anti-inflammatory cytokines.

The progesterone dose of 100 mg injected subcutaneously, as described in the examples, was based on the previously demonstrated observation that a subcutaneous formulation, commercially available for use in fertility treatment outside the United States (FDA IND 102771), achieves rapid, reliable progesterone serum concentrations approximating the luteal phase of the menstrual cycle. We aimed to target a progesterone level between that of the luteal phase and pregnancy, the latter of which can be as high as 290 ng/mL. Although data on outcomes of pregnant women with COVID-19 remain inconclusive, some reports have suggested that the pulmonary disease in pregnant women may be comparable, if not milder, than in age-matched nonpregnant female control subjects. This may be partly due to decreased production of proinflammatory factors inherent in pregnancy. To maintain our target progesterone level, the dose was administered twice daily for up to 5 days. Daily serum measurements confirmed the rapid increase in and sustained levels of progesterone; as expected, levels ranged between those seen in the luteal phase of menstrual cycle and the third trimester of pregnancy.

A major concern about exogenous sex hormone administration is the development of thrombotic disease; particularly when coupled with a disease already known for its coagulopathic effects.

This risk is most prominent in women who receive estrogen-containing contraceptives and appears to be most related to estrogen dose. In fact, progesterone-only contraceptives do not confer an increased risk of venous thromboembolic disease. Even IV progesterone, as used in phase 3 clinical trials of traumatic brain injury, was not associated with increased risk of thromboembolic disease. Nonetheless, all patients in our study received prophylactic-dose anticoagulation. We similarly observed that use of progesterone was safe overall and not associated with any significant increase in the risk of thromboembolism.

This trial showed very encouraging outcome data, showing that administration of progesterone at a dose of 100 mg twice daily by subcutaneous injection represents a safe and effective approach to the treatment of COVID-19 by improving the clinical status among men with moderate to severe illness. While the studies described herein treated male subjects, the embodiments of the present invention are not limited male subjects. Accordingly, progesterone treatment can be administered to female subjects as well.

As such, various embodiments of the present invention are based, at least in part, on these findings.

Various embodiments of the present invention provide for a method of treating coronavirus disease 2019 (COVID-19) in a subject in need thereof, comprising: administering a quantity of a progesterone receptor agonist to the subject. COVID-19 can be caused by SARS-CoV-2 or a variant of SARS-CoV-2. Variants of SARS-CoV-2 can include but are not limited to variants of interest and variants of concern, as defined and/or identified by the World Health Organization (WHO). SARS-CoV-2 variants of concerns currently include but are not limited to Alpha (B.1.1.7), Beta (B.1.351, B.1.351.2, B.1.351.3), Delta (B.1.617.2, AY.1, AY.2, AY.3), and Gamma (P.1, P.1.1, P.1.2).

In various embodiments, the quantity of the progesterone receptor agonist is about 50 mg to about 150 mg. In various embodiments, the quantity of progesterone receptor agonist is about 25 mg-50 mg. In various embodiments, the quantity of progesterone receptor agonist is about 50 mg-75 mg. In various embodiments, the quantity of progesterone receptor agonist is about 75 mg-100 mg. In various embodiments, the quantity of progesterone receptor agonist is about 100 mg-125 mg. In various embodiments, the quantity of progesterone receptor agonist is about 125 mg-150 mg.

In various embodiments, the quantity of progesterone receptor agonist results in a serum progesterone level of about 10 ng/mL-20 ng/mL in the subject. In various embodiments, the quantity of progesterone receptor agonist results in a serum progesterone level of up to 290 ng/mL in the subject. In various embodiments, the quantity of progesterone receptor agonist results in a serum progesterone level of about 11 ng/mL-288 ng/mL in the subject. In various embodiments, the quantity of progesterone receptor agonist results in a serum progesterone level of about 20 ng/mL-50 ng/mL in the subject. In various embodiments, the quantity of progesterone receptor agonist results in a serum progesterone level of about 51 ng/mL-100 ng/mL in the subject. In various embodiments, the quantity of progesterone receptor agonist results in a serum progesterone level of about 101 ng/mL-150 ng/mL in the subject. In various embodiments, the quantity of progesterone receptor agonist results in a serum progesterone level of about 151 ng/mL-200 ng/mL in the subject. In various embodiments, the quantity of progesterone receptor agonist results in a serum progesterone level of about 201 ng/mL-250 ng/mL in the subject. In various embodiments, the quantity of progesterone receptor agonist results in a serum progesterone level of about 251 ng/mL-300 ng/mL in the subject.

In various embodiments, the progesterone receptor agonist is selected from the group consisting of progesterone, levonorgestrel, etonogestrel, hydroxyprogesterone caproate, ulipristal acetate, medroxyprogesterone acetate, norethindrone, desogestrel, chlormadinone acetate, progesterone, dienogest, drospirenone, dydrogesterone, everolimus, hydroxyprogesterone, lynestrenol, medrogestone, medroxyprogesterone, megestrol acetate, nomegestrol acetate, norgestrel, promegestone, vilaprisan, danazol, methylestrenolone, VOLT-02, EC-313, MDC-WWM, and combinations thereof.

In various embodiments, the progesterone receptor agonist is administered twice daily. In various embodiments, the progesterone receptor agonist is administered once daily. In various embodiments, the progesterone receptor agonist is administered three time daily. In various embodiments, the progesterone receptor agonist is administered four time daily.

In various embodiments, the progesterone receptor agonist is administered subcutaneously. In various embodiments, the progesterone receptor agonist is administered subcutaneously, twice daily for five days.

In various embodiments, the progesterone receptor agonist is administered for up to 5 days. In various embodiments, the progesterone receptor agonist is administered for about 5 days. In various embodiments, the progesterone receptor agonist is administered for about 7 days. In various embodiments, the progesterone receptor agonist is administered for about 10 days. In various embodiments, the progesterone receptor agonist is administered for about 14 days. In various embodiments, the progesterone receptor agonist is administered for about 21 days. In various embodiments, the progesterone is administered for more than 21 days.

In various embodiments, the progesterone receptor agonist is administered in addition to one or more standard-of-care therapy for COVID-19.

In various embodiments, the method further comprises administering a concomitant therapeutic intervention. In various embodiments, the concomitant therapeutic intervention is selected from the group consisting of azithromycin, remdesivir, systemic glucocorticoid, tocilizumab, convalescent plasma, hydroxychloroquine, monoclonal antibodies against SARS-CoV-2, baricitinib, anticoagulant, and combinations thereof.

In various embodiments, systemic glucocorticoid is dexamethasone, prednisone, methylprednisolone, or combinations thereof.

In various embodiments, the monoclonal antibodies against SARS-CoV-2 is casirivimab, imdevimab, bamlanivimab, etesevimab, sotrovimab, or combinations thereof. In various embodiments, the monoclonal antibodies against SARS-CoV-2 is a combination of casirivimab and imdevimab. In various embodiments, the monoclonal antibodies against SARS-CoV-2 is a combination of bamlanivimab and etesevimab.

In various embodiments, the progesterone receptor agonist is progesterone. In various embodiments, the quantity of progesterone is about 100 mg. In various embodiments, the quantity of progesterone is about 50 mg-150 mg. In various embodiments, the quantity of progesterone is about 25 mg-50 mg. In various embodiments, the quantity of progesterone is about 50 mg-75 mg. In various embodiments, the quantity of progesterone is about 75 mg-100 mg. In various embodiments, the quantity of progesterone is about 100 mg-125 mg. In various embodiments, the quantity of progesterone is about 125 mg-150 mg.

In various embodiments, the quantity of progesterone results in a serum progesterone level of about 10 ng/mL-20 ng/mL in the subject. In various embodiments, the quantity of progesterone results in a serum progesterone level of up to 290 ng/mL in the subject. In various embodiments, the quantity of progesterone results in a serum progesterone level of about 11 ng/mL-288 ng/mL in the subject. In various embodiments, the quantity of progesterone results in a serum progesterone level of 11.1 ng/mL-290 ng/mL in the subject. In various embodiments, the quantity of progesterone results in a serum progesterone level of about 20 ng/mL-50 ng/mL in the subject. In various embodiments, the quantity of progesterone receptor in a serum progesterone level of about 51 ng/mL-100 ng/mL in the subject. In various embodiments, the quantity of progesterone results in a serum progesterone level of about 101 ng/mL-150 ng/mL in the subject. In various embodiments, the quantity of progesterone results in a serum progesterone level of about 151 ng/mL-200 ng/mL in the subject. In various embodiments, the quantity of progesterone results in a serum progesterone level of about 201 ng/mL-250 ng/mL in the subject. In various embodiments, the quantity of progesterone results in a serum progesterone level of about 251 ng/mL-300 ng/mL in the subject.

In various embodiments, the quantity of progesterone is administered twice a day. In various embodiments, the quantity of progesterone is administered once a day. In various embodiments, the quantity of progesterone is administered three time a day. In various embodiments, the quantity of progesterone is administered four times a day.

In various embodiments, the subject is male. In various embodiments, the subject is male who has one or more symptoms of COVID-19 or is confirmed to have COVID-19. In various embodiments, the subject is male who has one or more symptoms of COVID-19 or is confirmed to have COVID-19, and is hospitalized.

In various embodiments, the subject has or is suspected to have a baseline serum progesterone level of less than 1 ng/mL. In various embodiments, the subject has or is suspected to have a baseline progesterone level of less than 5 ng/mL. In various embodiments, the subject has or is suspected to have a baseline progesterone level of less than 10 ng/mL. In various embodiments, the subject has or is suspected to have a baseline progesterone level of less than 15 ng/mL. In various embodiments, the subject has or is suspected to have a baseline progesterone level of less than 20 ng/mL. In various embodiments, the subject has or is suspected to have a baseline progesterone level of less than 25 ng/mL. In various embodiments, the subject has or is suspected to have a baseline progesterone level of less than 290 ng/mL.

In various embodiments, the subject has or is suspected to have a baseline progesterone level between 1 ng/mL-10 ng/mL. In various embodiments, the subject has or is suspected to have a baseline progesterone level between 10 ng/mL-20 ng/mL. In various embodiments, the subject has or is suspected to have a baseline progesterone level between 21 ng/mL-50 ng/mL. In various embodiments, the subject has or is suspected to have a baseline progesterone level between 51 ng/mL-100 ng/mL. In various embodiments, the subject has or is suspected to have a baseline progesterone level between 101 ng/mL-150 ng/mL. In various embodiments, the subject has or is suspected to have a baseline progesterone level between 151 ng/mL-200 ng/mL. In various embodiments, the subject has or is suspected to have a baseline progesterone level between 201 ng/mL-250 ng/mL. In various embodiments, the subject has or is suspected to have a baseline progesterone level between 251 ng/mL-300 ng/mL.

In various embodiments, the subject has or is suspected to have a progesterone level of less than 11 ng/mL. In various embodiments, the subject has or is suspected to have a progesterone level of less than 288 ng/mL.

Baseline progesterone level refers to the progesterone level prior to treatment with a progesterone receptor antagonist in accordance with various embodiments of the present invention.

In various embodiments, the method comprises subcutaneously administering about 100 mg of progesterone twice a day to a male subject who has one or more symptoms of COVID-19, or who is confirmed to have COVID-19. In various embodiments, the progesterone is administered for up to 5 days. In various embodiments, the progesterone is administered for about 5 days. In various embodiments, the progesterone is administered for about 7 days. In various embodiments, the progesterone is administered for about 10 days. In various embodiments, the progesterone is administered for about 14 days. In various embodiments, the progesterone is administered for about 21 days. In various embodiments, the progesterone is administered for more than 21 days.

In various embodiments of the invention, the quantity of the progesterone receptor agonist or progesterone for use with the methods described herein may be in the range of 1-5 units/kg, 5-10 units/kg, 10-50 units/kg, 50-100 units/kg, 100-150 units/kg, 150-200 units/kg, 100-200 units/kg, 200-300 units/kg, 300-400 units/kg, or 400-500 units/kg. In some embodiments, the quantity of the progesterone agonist or progesterone is about 25-50 units/kg, about 50-75 units/kg, or about 75-100 units/kg.

In some embodiments of the invention, the quantity of the progesterone receptor agonist or progesterone can be in the range of about 1-5 μg/day, 5-10 μg/day, 10-15 μg/day, 15-20 μg/day, 10-20 μg/day, 20-30 μg/day, 30-40 μg/day, 40-50 μg/day, 50-60 μg/day, 60-70 μg/day, 70-80 μg/day, 80-90 μg/day, 90-100 μg/day, 100-110 μg/day, 110-120 μg/day, 120-130 μg/day, 130-140 μg/day, 140-150 μg/day, 150-160 μg/day, 160-170 μg/day, 170-180 μg/day, 180-190 μg/day, 190-200 μg/day, 200-210 μg/day, 210-220 μg/day, 220-230 μg/day, 230-240 μg/day, 240-250 μg/day, 250-260 μg/day, 260-270 μg/day, 270-280 μg/day, 280-290 μg/day or 290-300 μg/day.

In some embodiments of the invention, the quantity of the progesterone receptor agonist or progesterone can be in the range of about 10-50 μg/day, 50-100 μg/day, 100-150 μg/day, 150-200 μg/day, 100-200 μg/day, 200-300 μg/day, 300-400 μg/day, 400-500 μg/day, 500-600 μg/day, 600-700 μg/day, 700-800 μg/day, 800-900 μg/day, 900-1000 μg/day, 1000-1100 μg/day, 1100-1200 μg/day, 1200-1300 μg/day, 1300-1400 μg/day, 1400-1500 μg/day, 1500-1600 μg/day, 1600-1700 μg/day, 1700-1800 μg/day, 1800-1900 μg/day, 1900-2000 μg/day, 2000-2100 μg/day, 2100-2200 μg/day, 2200-2300 μg/day, 2300-2400 μg/day, 2400-2500 μg/day, 2500-2600 μg/day, 2600-2700 μg/day, 2700-2800 μg/day, 2800-2900 μg/day or 2900-3000 μg/day.

In some embodiments of the invention, the quantity of the progesterone receptor agonist or progesterone can be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day.

In various embodiments, the quantity of the progesterone receptor agonist or progesterone can be about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg/day, or a combination thereof. In various embodiments, the quantity of the progesterone agonist or progesterone can be about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg/day, or a combination thereof. Here, “μg/kg/day” or “mg/kg/day” refers to μg or mg agent per kg body weight of the subject per day.

In various embodiments, the present invention provides pharmaceutical compositions including a pharmaceutically acceptable excipient along with a quantity of a progesterone agonist or progesterone. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like.

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

The term “prodrug” refers to compounds that are rapidly transformed in vivo to yield the functionally active one or more peptides as disclosed herein or a mutant, variant, analog or derivative thereof. As used herein, a prodrug is a compound that, upon in vivo administration, is metabolized or otherwise converted to the biologically, pharmaceutically or therapeutically active form of the compound. A prodrug of the one or more peptides as disclosed herein or a mutant, variant, analog or derivative thereof can be designed to alter the metabolic stability or the transport characteristics of one or more peptides as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more peptides as disclosed herein or a mutant, variant, analog or derivative thereof, those of skill in the pharmaceutical art generally can design prodrugs of the compound. Suitable examples of prodrugs include methyl, ethyl and glycerol esters of the corresponding acid.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective progesterone agonist or progesterone can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied tissue, or the responses observed in the appropriate animal models, as previously described.

### Kits

The present invention is also directed to a kit to treat COVID-19. The kit is useful for practicing the inventive method of treating COVID-19. The kit is an assemblage of materials or components, including at least one of the inventive compositions. Thus, in some embodiments the kit contains a composition including a progesterone agonist or progesterone as described above.

The exact nature of the components configured in the inventive kit depends on its intended purpose. For example, some embodiments are configured for the purpose of treating COVID-19. In one embodiment, the kit is configured particularly for the purpose of treating mammalian subjects. In another embodiment, the kit is configured particularly for the purpose of treating human subjects. In another embodiment, the kit is configured particularly for the purpose of treating human male subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat COVID-19. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in treatment of infectious diseases or for subcutaneous administration. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing the progesterone agonist or the progesterone. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Study Population

Selection of the Study Population—Adult (≥18 years old) genetic male patients with laboratory-confirmed COVID-19 hospitalized at Cedars-Sinai Medical Center.

Inclusion Criteria
1. Hospitalized adult (≥18 years old) genetic male patients
2. Laboratory-confirmed COVID-19 as determined by PCR, or other commercial or public health assay, as documented by either of the following:
   a. in any specimen <72 hours prior to randomization
   b. in any specimen collected ≥72 hours prior to randomization, with progressive disease suggestive of ongoing COVID-19 infection.
3. Subject (or legally authorized representative) provides written informed consent prior to initiation of any study procedures
4. Understands and agrees to comply with planned study procedures
5. Agrees to the collection of venous blood per protocol
6. Illness of any duration and at least one of the following: Radiographic infiltrates by imaging (chest x-ray, CT scan, etc.), or clinical assessment (evidence of rales/crackles on exam) AND SpO₂≤94% on room air or requiring supplemental oxygen
7. Must agree to be placed on anticoagulation for prevention of deep venous thrombosis (DVT) while hospitalized
8. Must agree to use suitable barrier method of contraception for the duration of the study

Exclusion Criteria
1. ALT/AST>5 times the upper limit of normal
2. History of thromboembolic disease
3. History of breast cancer
4. Allergy to progesterone and betcyclodextrin
5. History of seizure disorder
6. Use of supplemental oxygen prior to hospital admission
7. Requiring higher than 50% supplemental oxygen by high flow nasal cannula or mechanical ventilation
8. Enrolled in any other interventional clinical trials for COVID-19

While these are exclusion criteria for the clinical trial study, these exclusion criteria do not preclude embodiments of the present invention to encompass patients who exhibit one or more of these criteria unless specifically disclaimed in the claims.

All participants, while hospitalized, were required to be receiving thromboembolism chemoprophylaxis (subcutaneous unfractionated heparin [5,000 units twice daily] or enoxaparin [40 mg daily]). Contraindications to anticoagulants precluded study enrollment. Patients were excluded if they had a history of thromboembolic disease, breast cancer, or liver transaminases greater than five times the upper limit of normal.

Subject Screening and Enrollment: Because COVID-19 is a reportable disease, hospital epidemiology knows about every patient under investigation and confirmed case that occurs in Cedars-Sinai Medical Center. Screening will be conducted by any study team member who has access to the hospital epidemiology COVID-19 case list. They study team, will then screen the patients based on the stated inclusion and exclusion criteria and potential participants will be identified.

Subject Recruitment

They study team, will then screen the patients based on the stated inclusion and exclusion criteria and potential participants will be identified.

It is anticipated that patients with COVID-19 will present to participating hospitals, and that no external recruitment efforts towards potential subjects are needed. Recruitment efforts may also include dissemination of information about this trial to other medical professionals/hospitals.

Patients that are confirmed to have SARS-CoV-2 will be assessed for eligibility.

Screening will begin with a brief discussion with study staff. Some will be excluded based on demographic data and medical history, <18 years of age, renal failure, etc. Information about the study will be presented to potential subjects (or legally authorized representative) and questions will be asked to determine potential eligibility. Screening procedures can begin only after informed consent is obtained.

### Study Design and Methods

This study is a phase 1b, pilot, randomized control trial to evaluate safety and clinical efficacy of progesterone in comparison to standard of care (SOC) in hospitalized men with COVID-19.

We believe that progesterone confers protection to women by reducing the inflammatory surge that leads to lung injury in COVID-19. There is a subcutaneous formulation of progesterone which allows for higher and more predictable serum concentration. This formulation is currently commercially available outside of United States for use in fertility treatment. There is an active Investigational New Drug (IND) application with the Food and Drug Administration for fertility treatments using this formulation.

In our trial, 40 subjects will be randomized, 20 will receive institutional standard of care (SOC) and 20 will receive progesterone 100 mg subcutaneously twice daily for five days in addition to SOC.

Subjects will be assessed daily while hospitalized. In the event of clinical deterioration or at Day 7 and absence of clinical improvement, subjects in the control group may receive treatment with study product. Subjects may be withdrawn from active participation at day 7 or clinical deterioration requiring mechanical ventilation whichever comes first. This is to allow subjects to enroll in other trials should their condition not improve. Follow-up is for approximately 15 days. Withdrawn subjects will also be assessed at day 15. Discharged patients will be asked to participate in a phone or video study visits on Days 7 and 15.

All subjects will undergo a series of efficacy, safety, and laboratory assessments. Research Blood samples will be obtained on Day 1-5. Routine care and safety monitoring labs including White cell count (WBC), hemoglobin, platelets, creatinine, glucose, total bilirubin, alanine transaminase (ALT), and aspartate transaminase (AST) will be collected on Days 1-5, 7 and 15 (only while hospitalized) if they are performed as part of standard of care. In the control subjects who deteriorate and receive treatment with study drug, research labs (progesterone level and cytokine sample) will be obtained for 5 days. The standard of care safety labs will be collected during the five days of treatment if drawn as part of standard of care.

The change in clinical status of subjects at Day 7 will be assessed based on the following 7-point ordinal scale:
1. Death
2. Hospitalized, on invasive mechanical ventilation or extracorporeal membrane oxygenation (ECMO)
3. Hospitalized, on high flow oxygen devices
4. Hospitalized, requiring supplemental oxygen
5. Hospitalized, not requiring supplemental oxygen
6. Not hospitalized, limitation on activities
7. Not hospitalized, no limitations on activities

Secondary endpoints will include change in ordinal outcome assessed daily while hospitalized and on Day 15, National Early Warning Score (NEWS) assessed daily while hospitalized and on Days 7 and 15, if available. Also, duration of supplemental oxygen, mechanical ventilation, and hospitalization are included in secondary endpoints.

Death, serious adverse events (SAE's), and grade 3 and 4 adverse events (AEs) are will be assessed and reported throughout the study.

Routine care and safety monitoring labs will include White blood cell count (WBC), hemoglobin, platelets, creatinine, glucose, total bilirubin, alanine transaminase (ALT), aspartate transaminase (AST). These labs will be collected on Days 1-5, 7 or 15 (while hospitalized) only if obtained as part of standard of care. Any inflammatory markers such as D-dimer, C-reactive protein, ferritin level or IL-6 levels that are obtained as part of standard of care during the study will also be collected.

Standard of care safety labs that are obtained on control subjects who deteriorate and receive treatment with study drug will also be collected during the five days of treatment. Research labs will again get collected on these patients.

Specimen collection: Blood will be collected in conjunction with scheduled blood draws required for medical necessity and delivered to the biobank for processing and storage.

Specimen Processing
Plasma and serum will be aliquoted into 500 ul Eppendorf tubes.
All specimens will be labeled with labeled with a unique subject number that is generated after entering the patient information into a REDCap database.
Blood specimens will be stored in a −80° freezer in (SSB110).

The PI will be responsible for the storage and care of the specimens. The specimens will be kept indefinitely until all the material is used up.

| | | | | Day 7^{e} | Day 15^{e} | |
|---|---|---|---|---|---|---|
| | | | | (±1 days) | (±1 days) | |
| | | | | In-hospital, | In-hospital, | |
| | | | | phone visit, | phone visit, | |
| | | | Daily, until | or | or | |
| | Screening | Day | hospital | outpatient | outpatient | Early |
| Procedures | Visit | 1^{b} | discharge | visit | visit | Termination |
| Informed Consent | X | | | | | |
| Inclusion & exclusion criteria | X | X | | | | |
| Randomization to receive study | X | | | | | |
| drug or no study drug^{g} | | | | | | |
| Data collection: Demographics | X | | | | | |
| Data collection: Medical history | X | | | | | |
| Data collection: Current and | X | X | | | | |
| prior procedures/medications | | | | | | |
| Targeted Physical Examination^{f} | X | | | | | |
| Height, Weight | X | | | | | |
| Vital signs | | X | X | X | X | X |
| | | | (Daily) | | | |
| SpO2 (estimate of oxygen in the | | X | X | X | X | X |
| blood) | | | (Daily) | | | |
| Clinical and safety laboratory | | X | X | X (while | X (while | X (while |
| sampling^{c} | | | (Days 2-5 | hospitalized) | hospitalized) | hospitalized) |
| | | | while | | | |
| | | | hospitalized) | | | |
| Data collection: Assessment of | | X | X | X | X | X |
| supplemental oxygen and | | | (Daily) | | | |
| mechanical ventilation | | | | | | |
| requirements | | | | | | |
| Clinical status assessment | | X | X | X | X | X |
| | | | (Daily) | | | |
| NEWS (National Early Warning | | X | X | X | X | X |
| Score) assessment of degree of | | | (Daily) | | | |
| illness | | | | | | |
| Study drug (if randomized to | | X | X | | | |
| receive study drug) | | | | | | |
| Administration of study drug | | X | X | | | |
| | | | (Days 2-5, | | | |
| | | | while | | | |
| | | | hospitalized) | | | |
| Adverse events (AEs) | | X | X | X | X | X |
| assessment and data collection | | | (Daily) | | | |
| concomitant | | | | | | |
| procedures/therapies/medications | | X | RX | | | |
| Progesterone and cytokine blood | | | (Days 2-5, | | | |
| samples | | | while | | | |
| | | | hospitalized) | | | |

^{a}Screening Visit to occur on the day of or one day before first administration of study drug (Day −1 or 1)
^{b}All procedures should be performed prior to study drug administration with the exception of AEs and concomitant procedures/therapies/medications data. Lab results within 48 hours of Screening or Day 1 is acceptable
^{c}Clinical laboratory sampling includes chemistry and hematology panels will be collected only if drawn as part of standard of care.
^{d}Study drug to be administered twice daily
^{e}May be a phone visit(for discharged patients); Procedures will be performed if patient available for clinic visit; If patient is in-house, information will be collected via chart review.
^{f}Physical exam obtained between admission and Day 1 is acceptable.
^{g}In the event of clinical deterioration or at Day 7 and absence of clinical improvement, subjects in the control group may receive treatment with study product. These subjects will have progesterone and cytokine samples again collected.

### Data Collection and Management

Data Procurement: The study team and study coordinators/nurse, will be responsible for data procurement from the medical chart. After the patient has provided informed consent to join the study, study staff will create a subject profile within a REDCap database, which will generate a subject identifier. The study team members will be the only ones that will have access to this information that is stored in a password protected file on a secure server behind a CSMC firewall. Chart review throughout hospitalization will be conducted and periodic chart review up to a year will be performed to determine the patient status.

### Time Period of Data Under Review

Data will be collected daily during admission to monitor laboratory and clinical status as well as safety. Follow-up data will be collected as available. Information will be kept for an indefinite amount of time.

Variables collected: The following data points/variables will be collected:
Demographics
Medical history
Vital signs
Imaging
Progress information
Lab results
Hospital/ICU assessments
Hospital course and discharge location
Collection of data from patient charts to evaluate overall outcome.

Source documents: CS-link will be the source of all the data. The information will be directly entered into the REDCap database for long-term storage.

Data Collection and Storage: All data are collected and stored electronically behind a password and firewall-protected CSMC server. No hardcopies will be recorded. Only the PI, and study staff will have access to the data and link to PHI. The PHI will be removed from the database 1 year after enrollment. However, the HIPAA-limited data will be kept indefinitely in the REDCap database and maintained by the PI.

Confidentiality and Security of Data: To minimize the potential for risks related to a breach of confidentiality or research data:
Only the PI and delegated study staff will have access to PHI.
All samples will be labeled with a unique subject identifier. All data provided to collaborators will be labeled with the subject identifier with no reference to PHI.
Electronic research records will not be stored on a researcher's private computer, laptop or portable device unless these devices are encrypted and approved by the EIS. Research data and patient information may not leave Cedars-Sinai except for legitimate work-related purposes and in accordance with rules for off-campus transport of such information.

### Data and Safety Monitoring

Data and Safety Monitoring Plan
A Data Monitoring Committee (DMC) will be assembled to assess safety endpoints. The committee will be comprised of the study biostatistician and 1 non-study personnel who is an expert in the field of Pulmonary Medicine. The DMC will not have competing or any financial interests.
The committee will meet for an interim analysis after the first 10 patients, 5 in each arm, complete Day 15 of the study.
A rigorous screening process will be utilized to verify eligibility.
During study drug treatment period, the subject will be monitored, and treatment will be discontinued at the PI's discretion.
At the PI or treating provider's discretion, subjects may be removed from active participation if their condition deteriorates requiring mechanical intubation to allow them to participate in other studies or receive other courses of treatment.
During the follow-up period, changes in health status will be evaluated by the study doctor and reported as deemed appropriate.

Quality control and quality assurance: Through the combination of our use of REDCap with its electronic error detection, QA/QC plan, and regular site monitoring, we will ensure the quality and completeness of data in this trial.

### Statistical Considerations

Study Outcome Measures: The primary outcome measure is change (Delta) in clinical status (from baseline) at Day 7 on the following 7-point ordinal scale:
1=Death
2=Hospitalized, on invasive mechanical ventilation or extracorporeal membrane oxygenation (ECMO)
3=Hospitalized, on high flow oxygen devices
4=Hospitalized, requiring supplemental oxygen
5=Hospitalized, not requiring supplemental oxygen
6=Not hospitalized, limitation on activities
7=Not hospitalized, no limitations on activities

In the event that a subject assigned to SOC is given study product due to clinical deterioration, the primary outcome will be imputed at Day & as the subject's last clinical assessment prior to receiving study product.

The above Delta will be condensed into the following 3-point ordinal scale, Delta3:
1=Patient status worsened (for example went from level 4 to level 3 on the 7-point scale)
2=Patient stayed the same
3=Patient improved (for example went from level 2 to level 5 on the 7-point scale)

The above Delta3 will be further condensed into a binary outcome variable: Worsened versus {Stayed the same or Improved}.

Delta, Delta3, and the binary Worsened variable will be summarized by frequency and percent in each group (SOC+Progesterone and SOC).

Promising/Favorable Study Result: A shift in the Delta or Delta3 or Worsened group distribution towards more improvement in the SOC+Progesterone group will be viewed as a promising/favorable study result.

Delta and Delta3 will be compared across the groups by the Wilcoxon rank sum test. Worsened will be compared across the groups by the Fisher exact test.

Continuous variables will be summarized by mean, standard deviation (SD), median, and range, and will be compared across groups by the independent samples t test and the Wilcoxon rank sum test, as appropriate. Categorical variables will be summarized by frequency and percentage and will be compared across groups by the Fisher exact test. Within group change on numerical variables will be assessed by the paired t test and the Wilcoxon signed rank test, as appropriate. Within group change on ordinal variables will be assessed by the Wilcoxon signed rank test. Within group change on binary variables will be assessed by McNemar's test for related proportions.

Sample Size Considerations: The study is a pilot study. If the result is favorable/promising, we plan to do a larger, definitive study. Parameter estimates from this pilot study will be used to power the definitive study. A sample size of 20 per group (total sample size=40) will provide estimates that are adequate for powering the definitive study.

### Example 2

The following are the summary and results of the study conducted as described by Example 1, above.

### Study Design

Approximately 40 subjects were to be randomized, with 1:1 allocation to institutional standard of care (SOC) versus progesterone 100 mg subcutaneously twice daily for five days in addition to SOC. Subjects were assessed daily while hospitalized. In the event of clinical deterioration or at Day 7 and absence of clinical improvement, SOC subjects received treatment with study product. These patients remained in their intention-to-treat group for purpose of analysis.

Patients who had sufficiently improved, in the judgment of the treating providers, could be discharged from the hospital before completing their assigned courses of treatment. The protocol permitted use of other agents with presumptive activity against SARS-CoV-2 if such use was part of institutional SOC.

The protocol was amended to include patients with chronic kidney disease, based on an FDA general recommendation to COVID-19 clinical trials to consider inclusion of at-risk populations for severe illness. The study period was shortened to 15 days from the initial 29 days to allow enrolled patients with progressive illness to participate in other investigational trials without the need to withdraw from this study. Because of a shortage of SARS-CoV-2 PCR testing supplies, an amendment was added to allow enrollment of patients with a positive PCR test result before 72 h from the time of screening and clinical evidence of progressive disease. All subjects enrolled met the initial enrollment condition with a positive PCR test result within 72 h of screening. Protocol amendments were authorized and approved by the institutional review board and FDA.

Study patients were assessed daily for 15 days or until discharge, whichever came first. Discharged patients participated in phone or video study visits on days 7 and 15. Clinical assessment performed daily during hospitalization included evaluation of clinical status with daily vital signs, oxygen supplementation type and amount, need for mechanical ventilation, adverse events, and concomitant medications. WBCs, hemoglobin, platelets, electrolytes, BUN, creatinine, liver transaminases, and inflammatory markers, if obtained as part of SOC, were monitored on days 1 through 5, 7, and 15 while patients were hospitalized. Serum free and total progesterone levels were also measured on days 1 through 5. Self-reported race and ethnicity, obtained from medical records, were collected as demographic information to assess possible differences in disease severity or treatment response. Serious adverse events and grade 3 and 4 adverse events as described in the Division of AIDS Table for Grading the Severity of Adult and Pediatric Adverse Events were recorded.

Clinical status was assessed on a seven-point ordinal scale, similarly used by Goldman et al, as follows: 1, death; 2, hospitalized, on invasive mechanical ventilation or extracorporeal membrane oxygenation (ECMO); 3, hospitalized, on high-flow oxygen devices; 4, hospitalized, requiring supplemental oxygen; 5, hospitalized, not requiring supplemental oxygen; 6, not hospitalized, limitation on activities; and 7, not hospitalized, no limitations on activities. If the clinical status of a hospitalized patient changed on any given study day, the patient's worst clinical assessment score on the ordinal scale was documented.

### Primary Objective

Evaluate the safety and clinical efficacy of progesterone in comparison to SOC alone in hospitalized men with COVID-19.

### Primary Endpoint

In addition to safety and tolerability, the primary efficacy end point was change in patients' clinical status, assessed on a seven-point ordinal scale, from baseline to day 7. Secondary end points were hospital length of stay (LOS), days of supplemental oxygen use, and need for mechanical ventilation.

Change in clinical status of subjects at Day 7 compared to Baseline, based on the following 7-point ordinal scale:
1. Death
2. Hospitalized, on invasive mechanical ventilation or extracorporeal membrane oxygenation (ECMO)
3. Hospitalized, on high flow oxygen devices
4. Hospitalized, requiring supplemental oxygen
5. Hospitalized, not requiring supplemental oxygen
6. Not hospitalized, limitation on activities
7. Not hospitalized, no limitations on activities

### Statistical Considerations

Baseline is defined as clinical status on Day 1, shortly after randomization for SOC subjects and prior to first administration of study product for the progesterone group. In the event that a subject assigned to SOC was administered study product due to clinical deterioration prior to Day 7, the Day 7 assessment is imputed as the subject's last clinical assessment prior to receiving study product. The Wilcoxon rank-sum test is used to compare the change in clinical status between the two groups.

At the completion of enrollment, 22 subjects were randomized to SOC and 20 to the progesterone group. Two subjects randomized to progesterone withdrew from the study prior to receiving their first dose. Nine SOC subjects were treated with progesterone due to clinical deterioration prior to Day 7 (6 subjects) or absence of clinical improvement by Day 7 (3 subjects).

One subject assigned to the progesterone group had repeated non-compliance to the protocol and was transferred to another hospital at Day 5. For purpose of safety evaluation, follow up revealed that this subject died due to complications of disseminated cryptococcal infection. This subject has been excluded from analysis.

### Post Hoc Analyses

Because several patients in both groups experienced clinical deterioration over days 2 through 6 a sensitivity analysis was performed, considering the patients' worst status before day 7 as their baseline to capture the illness severity while assessing the change in clinical status score between the two groups. In another sensitivity analysis, for control patients who crossed over before day 7, their last clinical assessment before receiving progesterone was imputed as the day 7 assessment.

### Results

The results presented in this report are based on an open clinical database and should be considered preliminary at this time. There are no meaningful differences observed in the demographics of the two groups (Table 1A). The results of the Wilcoxon rank-sum test (Table 2A) suggest that there is a greater degree of improvement in clinical status at Day 7 for subjects who received progesterone compared to those on SOC (p=0.003). Of the 6 subjects in SOC group who were treated with progesterone prior to day 7, five had the same or worse actual clinical status than the imputed one.

Since several subjects in both groups experienced clinical deterioration on Days 2-6, a sensitivity analysis is presented considering the subjects' worst status prior to Day 7 (or prior to the imputed Day 7 for SOC subjects who were administered treatment) as their baseline in order to capture the severity of illness. The progesterone group continues to show greater improvement over the SOC group under the considerations of this sensitivity analysis (p<0.001). Five out of six subjects in SOC group who were treated with progesterone prior to day 7, had an imputed clinical status that was the same or worse than their actual clinical status at day 7.

TABLE 1A
Demographics and Baseline Characteristics (with N = 39 for all subjects)

| | | | Standard |
|---|---|---|---|
| | All Subjects | Progesterone | of Care |
| | N = 39 | N = 17 | N = 22 |
| Characteristic | n (%) | n (%) | n (%) |
| Age (years) [Mean (SD)] | 55.8 (16.23) | 57.4 (16.84) | 54.6 (16.05) |
| Body Mass Index (kg/m²) [Mean (SD)] | 31.9 (9.53) | 32.4 (11.21) | 31.4 (8.25) |
| Race [n (%)] | | | |
| Asian | 1 (2.6) | 0 (0.0) | 1 (4.5) |
| Black or African American | 3 (7.7) | 1 (5.9) | 2 (9.1) |
| Native Hawaiian or Other Pacific Islander | 1 (2.6) | 1 (5.9) | 0 (0.0) |
| White | 31 (79.5) | 12 (70.6) | 19 (86.4) |
| Other | 3 (7.7) | 3 (17.6) | 0 (0.0) |
| Ethnicity [n (%)] | | | |
| Hispanic or Latino | 24 (61.5) | 10 (58.8) | 14 (63.6) |
| Not Hispanic or Latino | 15 (38.5) | 7 (41.2) | 8 (36.4) |
| Number of Comorbidities [n (%)] | | | |
| None | 10 (25.6) | 6 (35.3) | 4 (18.2) |
| One | 19 (48.7) | 8 (47.1) | 11 (50.0) |
| Two or more | 10 (25.6) | 3 (17.6) | 7 (31.8) |
| Comorbidities [n (%)] | | | |
| Hypertension^{a} | 18 (46.2) | 7 (41.2) | 11 (50.0) |
| Diabetes^{a} | 8 (20.5) | 3 (17.6) | 5 (22.7) |
| Obesity^{b} | 18 (46.2) | 6 (35.3) | 12 (54.5) |

^{a}Reported in medical history
^{b}Defined as body mass index (BMI) greater than or equal to 30 kg/m²

TABLE 2A
Summary of Clinical Status at Day 7

| | | Standard | Proges- |
|---|---|---|---|
| | | of Care^{a} | terone |
| | | N = 22 | N = 17 |
| Parameter | Clinical Status | n (%) | n (%) |
| Baseline Status | 3-Hospitalized; on high flow nasal cannula | 0 (0.0) | 3 (17.6) |
| | 4-Hospitalized; requiring supplemental | 20 (90.9) | 10 (58.8) |
| | oxygen (not HFNC) | | |
| | 5-Hospitalized; not requiring supplemental oxygen | 2 (9.1) | 4 (23.5) |
| Worst Status Prior to Day 7 | 2-Hospitalized; on invasive mechanical | 2 (9.1) | 0 (0.0) |
| | ventilation or ECMO | | |
| | 3-Hospitalized; on high flow nasal cannula | 5 (22.7) | 5 (29.4) |
| | 4-Hospitalized; requiring supplemental oxygen | 13 (59.1) | 11 (64.7) |
| | (not HFNC) | | |
| | 5-Hospitalized; not requiring supplemental oxygen | 2 (9.1) | 1 (5.9) |
| Status at Day 7 | 2-Hospitalized; on invasive mechanical | 2 (9.1) | 0 (0.0) |
| | ventilation or ECMO | | |
| | 3-Hospitalized; on high flow nasal cannula | 5 (22.7) | 2 (11.8) |
| | 4-Hospitalized; requiring supplemental oxygen | 8 (36.4) | 2 (11.8) |
| | (not HFNC) | | |
| | 5-Hospitalized; not requiring supplemental oxygen | 4 (18.2) | 4 (23.5) |
| | 6-Not hospitalized; limitations on activities | 3 (13.6) | 7 (41.2) |
| | 7-Not hospitalized; no limitations on activities | 0 (0.0) | 2 (11.8) |
| Change from Baseline at Day 7 | −2 | 2 (9.1) | 0 (0.0) |
| | −1 | 5 (22.7) | 2 (11.8) |
| | 0 | 9 (40.9) | 3 (17.6) |
| | +1 | 4 (18.2) | 3 (17.6) |
| | +2 | 2 (9.1) | 7 (41.2) |
| | +3 | 0 (0.0) | 2 (11.8) |
| | P-value^{b} | 0.003 | |
| Change from Worst at Day 7 | 0 | 16 (72.7) | 3 (17.6) |
| | +1 | 4 (18.2) | 4 (23.5) |
| | +2 | 2 (9.1) | 8 (47.1) |
| | +3 | 0 (0.0) | 2 (11.8) |

| | |
|---|---|
| P-value^{b} | <0.001 |

^{a}For standard of care subjects who received study product due to clinical deterioration prior to Day 7, the Day 7 outcome is imputed as the subject′s last clinical assessment prior to receiving study product.
^{b}P-value is based on Wilcoxon rank-sum test

### Example 3

### Additional Discussion of the Results

Between April 27 and Aug. 5, 2020, 136 patients were screened and assessed for eligibility; 94 were deemed ineligible for the study. Of the 42 enrolled patients, 20 were randomized to the progesterone group and 22 to the control group. The trial completed enrollment, and the final follow-up for the last enrollee was on Aug. 20, 2020. Two patients in the progesterone group withdrew from the study before receiving progesterone and were excluded from analysis. Nine control patients were treated with progesterone because of clinical deterioration before day 7 (n=6, 27%) or absence of clinical improvement by day 7 (n=3, 14%).

One patient assigned to the progesterone group was repeatedly protocol-noncompliant and was transferred to another hospital on day 5 for insurance coverage reasons. For the purpose of safety evaluation, follow-up revealed that this patient died on day 7 of complications from disseminated cryptococcal infection in the setting of untreated HIV infection. All available data on this patient as obtained on days 1 through 5 of the study and clinical status on day 7 have been included in the analysis.

Because enrollment was faster than anticipated, the trial terminated recruitment soon after the interim safety analysis. After discussion with the data safety monitoring committee, further interim analyses were deemed unnecessary.

Demographics and baseline characteristics of the study population were balanced in the two study groups (Table 1B).

TABLE 1B
Baseline Characteristics (with N = 40 for all subjects)

| | All Subjects | Progesterone | Control Group |
|---|---|---|---|
| Characteristic | (N = 40) | Group (n = 18) | (n = 22) |
| Age, mean ± SD, y | 55.3 ± 16.4 | 56.0 ± 17.3 | 54.6 ± 16.0 |
| Baseline BMI, | 31.6 ± 9.5 | 31.9 ± 11.1 | 31.4 ± 8.3 |
| mean ± SD, kg/m2 | | | |
| Race, No. (%) | | | |
| White | 31 (77.5) | 12 (66.7) | 19 (86.4) |
| Black/African American | 4 (10.0) | 2 (11.1) | 2 (9.1) |
| Asian/Pacific Islander | 2 (5.0) | 1 (5.6) | 1 (4.5) |
| Other | 3 (7.5) | 3 (16.7) | 0 (0.0) |
| Ethnicities, No. (%) | | | |
| Hispanic or Latino | 24 (60.0) | 10 (55.6) | 14 (63.6) |
| Not Hispanic or Latino | 16 (40.0) | 8 (44.4) | 8 (36.4) |
| Comorbidities, No. (%) | | | |
| Hypertension | 19 (47.5) | 7 (38.9) | 12 (54.5) |
| Diabetes | 10 (25.0) | 4 (22.2) | 6 (27.3) |
| Obesity | 18 (45.0) | 6 (33.3) | 12 (54.5) |

The patient population had an overall mean age of 55.3±16.4 years and a mean BMI of a 31.6±9.5 kg/m. Self-reported race and ethnicity indicated that most were white (78%) and Hispanic (60%). Most patients had comorbid conditions including hypertension, diabetes, obesity, or a combination of these. At baseline, there was no statistically significant difference in clinical status between the two groups with 85% of all patients requiring supplemental oxygen.

### Primary End Point

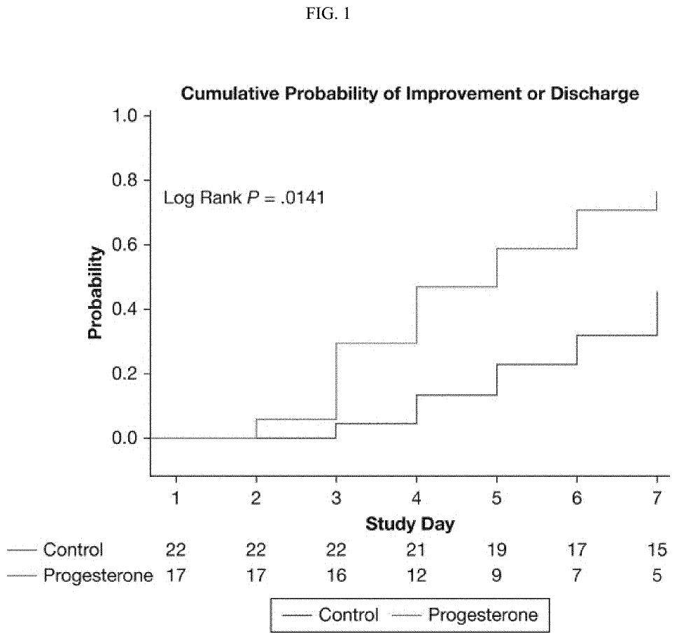

The primary outcome, ascertained as the overall change in clinical score status from baseline to day 7 on a seven-point ordinal scale, was a median of 1.5 points better for the progesterone group than the control group (95% CI, 0.0-2.0; P=0.024) (Table 2B). During the first seven study days, the cumulative probability of clinical improvement (an increase of at least one point on a seven-point scale or live discharge) was significantly higher in the progesterone group, 0.76 (95% CI, 0.55-0.93) vs 0.55 (95% CI, 0.28-0.68) in the control group (log-rank P=0.014), by Kaplan-Meier estimation. One patient in the progesterone group showed improvement on day 2 but was subsequently noncompliant with study protocols and was transferred to another facility. For the purpose of this Kaplan-Meier estimation, this subject was excluded (FIG. 1).

TABLE 2B
Clinical Status Based on Seven-Point Ordinal Scale

| | Progesterone Group | Control Group | P |
|---|---|---|---|
| | (n = 18) [No. (%)] | (n = 22) [No. (%)] | Value^{a} |
| Status at baseline, No. (%) | | | |
| 3: Hospitalized; on high-flow nasal | 3 (16.7) | 0 (0.0) | |
| cannula | | | |
| 4: Hospitalized; requiring supplemental | 11 (61.1) | 20 (90.9) | |
| oxygen (not HFNC) | | | |
| 5: Hospitalized; not requiring | 4 (22.2) | 2 (9.1) | |
| supplemental oxygen | | | |
| Status on day 7, No. (%) | | | |
| 1: Death | 1 (5.6) | 0 (0.0) | |
| 2: Hospitalized; on invasive mechanical | 0 (0.0) | 3 (13.6) | |
| ventilation or ECMO | | | |
| 3: Hospitalized; on high-flow nasal | 2 (11.1) | 3 (13.6) | |
| cannula | | | |
| 4: Hospitalized; requiring supplemental | 2 (11.1) | 8 (36.4) | |
| oxygen (not HFNC) | | | |
| 5: Hospitalized; not requiring | 4 (22.2) | 4 (18.2) | |
| supplemental oxygen | | | |
| 6: Not hospitalized; limitations on | 7 (38.9) | 4 (18.2) | |
| activities | | | |
| 7: Not hospitalized; no limitations on | 2 (11.1) | 0 (0.0) | |
| activities | | | |
| Change in status on day 7, No. (%) | | | |
| +3 | 2 (11.1) | 0 (0.0) | |
| +2 | 7 (38.9) | 3 (13.6) | |
| +1 | 3 (16.7) | 4 (18.2) | |
| 0 | 3 (16.7) | 9 (40.9) | |
| −1 | 2 (11.1) | 3 (13.6) | |
| −2 | 0 (0.0) | 3 (13.6) | |
| −3 | 1 (5.6) | 0 (0.0) | |
| Change in status on day 7, median | 1.5 (0.0 to 2.0) | 0.0 (−1.0 to 1.0) | .024 |
| (IQR) | | | |
| Status on day 15, No. (%) | | | |
| 1: Death | 1 (5.6) | 1 (4.5) | |
| 2: Hospitalized; on invasive mechanical | 0 (0.0) | 2 (9.1) | |
| ventilation or ECMO | | | |
| 3: Hospitalized; on high-flow nasal | 0 (0.0) | 2 (9.1) | |
| cannula | | | |
| 4: Hospitalized; requiring supplemental | 1 (5.6) | 0 (0.0) | |
| oxygen (not HFNC) | | | |
| 5: Hospitalized; not requiring | 1 (5.6) | 1 (4.5) | |
| supplemental oxygen | | | |
| 6: Not hospitalized; limitations on | 8 (44.4) | 12 (54.5) | |
| activities | | | |
| 7: Not hospitalized; no limitations on | 7 (38.9) | 4 (18.2) | |
| activities | | | |
| Change in status on day 15, No. (%) | | | |
| +4 | 1 (5.6) | 0 (0.0) | |
| +3 | 7 (38.9) | 2 (9.1) | |
| +2 | 4 (22.2) | 14 (63.6) | |
| +1 | 4 (22.2) | 1 (4.5) | |
| 0 | 1 (5.6) | 0 (0.0) | |
| −1 | 0 (0.0) | 2 (9.1) | |
| −2 | 0 (0.0) | 2 (9.1) | |
| −3 | 1 (5.6) | 1 (4.5) | |
| Change in status on day 15, median | 2.0 (1.0 to 3.0) | 2.0 (1.0 to 2.0) | .150 |
| (IQR) | | | |

ECMO = extracorporeal membrane oxygenation; HFNC = high-flow nasal cannula; IQR = interquartile range.
^{a}Exact Wilcoxon rank-sum test.

### Post Hoc Analyses

In a sensitivity analysis comparing worst clinical status before day 7 with clinical status on day 7, the progesterone group improved a median of two points more than the control group (95% CI, 1.0-2.0; P=0.006). This analysis captures the illness severity while assessing the change in clinical status score between the two groups; again favoring the progesterone group.

In a sensitivity analysis in which the last clinical assessment on the seven-point ordinal scale before crossing over was imputed as the day 7 score, overall change in score from baseline to day 7 was a median of 1.5 points better for the progesterone group than the control group (95% CI, 0.0-2.0; P=0.010).

### Secondary End Points and Adverse Events

Among patients assigned to the progesterone group, the median number of days on supplemental oxygen was 4.5 (IQR, 2.0-6.0) compared with 7.5 (IQR, 6.0-11.0) in the control group, for a median difference of 3 days. By day 7, nine of 18 patients (50%) in the progesterone group remained hospitalized, compared with 19 of 22 patients (86%) in the control group. Patients in the progesterone group had a median LOS of 7.0 days (IQR, 4.0-9.0) whereas the control group had a median LOS of 9.5 days (IQR, 7.0-14.0). At study completion, one patient in the progesterone group remained hospitalized compared with five in the control group. Mechanical ventilation was initiated in four of 22 control patients (18%), three before day 7, compared with none in the progesterone group. Although we see evidence of improved clinical outcomes in patients receiving progesterone, with fewer days of hospitalization, and less need for supplemental oxygen or mechanical ventilation, differences between groups did not meet conventional levels of statistical significance.

Although the patients were analyzed on an intent-to-treat basis, notably one-half of the six control patients who crossed over because of clinical deterioration before day 7 progressed to require mechanical ventilation. Of those, one was successfully liberated from the ventilator before completion of the study. The remaining one-half crossed-over patients (n=3), despite a clear trajectory of decline, did not require mechanical ventilation and improved to discharge before completion of the study.

Administration of expanded-use access and other medications was allowed for both the control and intervention groups (Table 4). A larger percentage of the control group received remdesivir, systemic glucocorticoids, tocilizumab, and convalescent plasma, but these differences were not significant. A greater proportion but equal number of patients in the intervention arm received azithromycin, although this was also not significant.

TABLE 4
Concomitant Therapeutic Interventions

| | Progesterone Group | Control Group |
|---|---|---|
| Intervention | (n = 18) [No. (%)] | (n = 22) [No. (%)] |
| Azithromycin | 10 (55.6) | 10 (45.5) |
| Remdesivir | 9 (50.0) | 15 (68.2) |
| Systemic glucocorticoids | 9 (50.0) | 15 (68.2) |
| Dexamethasone | 7 (38.9) | 10 (45.5) |
| Tocilizumab | 1 (5.6) | 4 (18.2) |
| Convalescent plasma | 0 (0.0) | 2 (9.1) |
| Hydroxychloroquine | 0 (0.0) | 1 (4.5) |

There were no serious adverse events, including life-threatening events, attributable to progesterone. There were two thromboembolic events in one patient (5.6%) in the progesterone group and two thromboembolic events in two patients (9.1%) in the control group (Table 3. Overall, there was no meaningful difference in the incidence of serious adverse events between the two groups.

TABLE 3
Serious Adverse Events by System Organ Class and Preferred Term

| | | | Control Group |
|---|---|---|---|
| | Progesterone | Control | After |
| | Group | Group | Progesterone^{a} |
| | (n = 18) | (n = 22) | (n = 9) |
| | [No. (%)] | [No. (%)] | [No. (%)] |
| Any SAE or death | 2 (11.1) | 5 (22.7) | 3 (33.3) |
| Blood and lymphatic system | | | |
| disorders | | | |
| Lymphocyte count decreased | 0 (0.0) | 1 (4.5) | 0 (0.0) |
| Cardiac disorders | | | |
| Cardiac arrest | 0 (0.0) | 1 (4.5) | 0 (0.0) |
| Hypoperfusion | 0 (0.0) | 3 (13.6) | 2 (22.2) |
| Renal and urinary disorders | | | |
| Creatinine increased | 0 (0.0) | 1 (4.5) | 0 (0.0) |
| Respiratory, thoracic, and | | | |
| mediastinal disorders | | | |
| Hypoxia | 0 (0.0) | 4 (18.2) | 3 (33.3) |
| Vascular disorders | | | |
| DVT | 1 (5.6) | 2 (9.1) | 1 (11.1) |
| Pulmonary embolism | 1 (5.6) | 0 (0.0) | 0 (0.0) |
| Death | 1 (5.6) | 1 (4.5) | 0 (0.0) |

SAE = serious adverse event.
^{a}For control patients who received progesterone due to clinical deterioration, this column represents SAEs that occurred after receiving progesterone.

There were two deaths, one in each group, during the total 15-day surveillance period, neither attributable to progesterone administration. There were no events requiring discontinuation of progesterone. For the control patients who crossed over, significant adverse events after progesterone administration are also listed in Table 3.

Serum progesterone levels were obtained at baseline and, as anticipated, were less than 1 ng/mL in all patients. After administration of two doses of subcutaneous progesterone, goal serum levels were achieved and maintained between 11.1 and 288 ng/mL on subsequent samples. Levels as high as 288 ng/mL, which can be seen during the third trimester of pregnancy, were tolerated well and not associated with any adverse events.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method of treating coronavirus disease 2019 (COVID-19) in a subject in need thereof, comprising:
administering a quantity of a progesterone receptor agonist to the subject.

2. The method of claim 1, wherein the progesterone receptor agonist is selected from the group consisting of progesterone, levonorgestrel, etonogestrel, hydroxyprogesterone caproate, ulipristal acetate, medroxyprogesterone acetate, norethindrone, desogestrel, chlormadinone acetate, progesterone, dienogest, drospirenone, dydrogesterone, everolimus, hydroxyprogesterone, lynestrenol, medrogestone, medroxyprogesterone, megestrol acetate, nomegestrol acetate, norgestrel, promegestone, vilaprisan, danazol, methylestrenolone, VOLT-02, EC-313, MDC-WWM, and combinations thereof.

3. The method of claim 1, wherein the quantity of the progesterone receptor agonist is about 50 mg to about 150 mg.

4. The method of claim 1, wherein the progesterone receptor agonist is progesterone.

5. The method of claim 4, wherein the quantity of progesterone is about 100 mg.

6. The method of claim 1, wherein the progesterone receptor agonist is administered one to four times daily.

7. The method of claim 1, wherein the progesterone receptor agonist is administered twice daily.

8. The method of claim 1, wherein the progesterone receptor agonist is administered subcutaneously.

9. The method of claim 1, wherein the progesterone receptor agonist is administered subcutaneously, twice daily for five days.

10. The method of claim 1, wherein the progesterone receptor agonist is administered in addition to one or more standard-of-care therapy for COVID-19.

11. The method of claim 1, wherein the subject is male.

12. The method of claim 1, wherein the subject has or is suspected to have a baseline serum progesterone level of less than 1 ng/mL.

13. The method of claim 1, wherein the subject has or is suspected to have a progesterone level of less than 20 ng/mL.

14. The method of claim 1, wherein after administration of the progesterone receptor agonist, the subject has a progesterone level of 11.1 ng/mL to 290 ng/mL.

15. The method of claim 1, wherein the subject is male who has one or more symptoms of COVID-19, is confirmed to have COVID-19, is hospitalized or combinations thereof.

16. The method of claim 1, comprising subcutaneously administering about 100 mg of progesterone twice a day to a male subject who has one or more symptoms of COVID-19, or who is confirmed to have COVID-19.

17. The method of claim 1, further comprising administering a concomitant therapeutic intervention selected from the group consisting of azithromycin, remdesivir, systemic glucocorticoid, tocilizumab, convalescent plasma, hydroxychloroquine, monoclonal antibodies against SARS-CoV-2, baricitinib, anticoagulant, and combinations thereof.

18. The method of claim 17, wherein systemic glucocorticoid is dexamethasone, prednisone, or methylprednisolone.

19. The method of claim 17, wherein the monoclonal antibodies against SARS-CoV-2 is casirivimab, imdevimab, bamlanivimab, etesevimab, sotrovimab, or combinations thereof, or wherein the monoclonal antibodies against SARS-CoV-2 is a combination of casirivimab and imdevimab, or a combination of bamlanivimab and etesevimab.

20. A method of treating coronavirus disease 2019 (COVID-19) in a male subject in need thereof, comprising:
administering about 100 mg of progesterone, twice daily to the male subject who has one or more symptoms of COVID-19, or who is confirmed to have COVID-19.
